(19) United States
(12) Patent Application Publication
(10) Pub. No.: US 2019/0290470 A1
(43) Pub. Date: Sep. 26, 2019

(54) Surgical Steady Chin Strap

(71) Applicant: Nancy Chen, Kapolei, HI (US)

(72) Inventor: Nancy Chen, Kapolei, HI (US)

(21) Appl. No.: 15/926,141

(22) Filed: Mar. 20, 2018

(51) Int. Cl. A61F 5/37

(52) U.S. Cl. CPC A61F 5/3707

(57) ABSTRACT

A surgical chin strap for stabilizing a patient's head and airway to improve oxygen delivery during surgery includes a chin cup, a first cranial strap, and a second cranial strap. The chin cup is terminally connected to the first cranial strap and the second cranial strap. The chin cup is secured about a patient's chin, while the free ends of the first cranial strap and the second cranial strap are affixed to a table. The first cranial strap includes a first fastener and a first plurality of restraining holes for securing the first cranial strap to the table. The second cranial strap includes a second fastener and a second plurality of restraining holes for securing the second cranial strap to the table. A third cranial strap may be used to stabilize the patient's forehead; the third cranial strap running perpendicular to the first cranial strap and the second cranial strap.

### FIELD OF THE INVENTION

The present invention relates generally to medical devices. More specifically, the present invention is a surgical chin strap for stabilizing a patient's head during surgery and improving the airway for oxygen delivery.

### BACKGROUND OF THE INVENTION

During surgery it is essential to stabilize the body of the patient in order to prevent any unintentional harm to the patient. Such stabilization is even more essential in surgeries carried out on or near vital organs. For eye surgery in particular, it is critical that the patient's head be stabilized to prevent involuntary movement of the patient's head. Unanticipated movement of the patient's head during such surgeries could lead to a laceration of the eye, which could lead to permanent damage of the patient's eye, such as blindness. A means for securely maintaining the position of a patient's head during surgery is needed to both assist surgeons during operation and to prevent undue injury to the patient. Additionally, it is critical that a patient's airway is stabilized during surgery while under local anesthesia to ensure the delivery of oxygen to the patient is uninhibited. Thus, a means for both stabilizing the patient's head and the patient's airway is needed to ensure the wellbeing of the patient during surgery.

Therefore it is an object of the present invention to provide a surgical steady chin strap for stabilizing a patient's head during surgery. Furthermore, the surgical steady chin strap stabilizes the patient's airway for oxygen delivery during surgery, which is essential for the success and wellbeing of the patient during any type of surgery under local anesthesia. The present invention includes a chin cup that is terminally connected to a first cranial strap and a second cranial strap. The chin cup is secured about the patient's chin, while the free ends of the first cranial strap and the second cranial strap are secured to the medical table in order to stabilize the patient's head. Both the first cranial strap and the second cranial strap provide a dual means for being secured to the medical table, to ensure the safety of the patient. The present invention may further include a third cranial strap that runs perpendicular to the first cranial strap and the second cranial strap. The third cranial strap is positioned about the patient's forehead to apply a downward force to the patient's head. Similar to the first cranial strap and the second cranial strap, the third cranial strap provides a dual means for being secured to the medical table, to ensure the safety of the patient.

### BRIEF DESCRIPTION OF THE DRAWINGS

FIG. 1 is a perspective view of the present invention.

FIG. 2 is a right side elevational view of the present invention.

FIG. 3 is a left side elevational view of the present invention.

FIG. 4 is a top plan view of the present invention.

FIG. 5 is a diagram showing the present invention secured about a patient's chin.

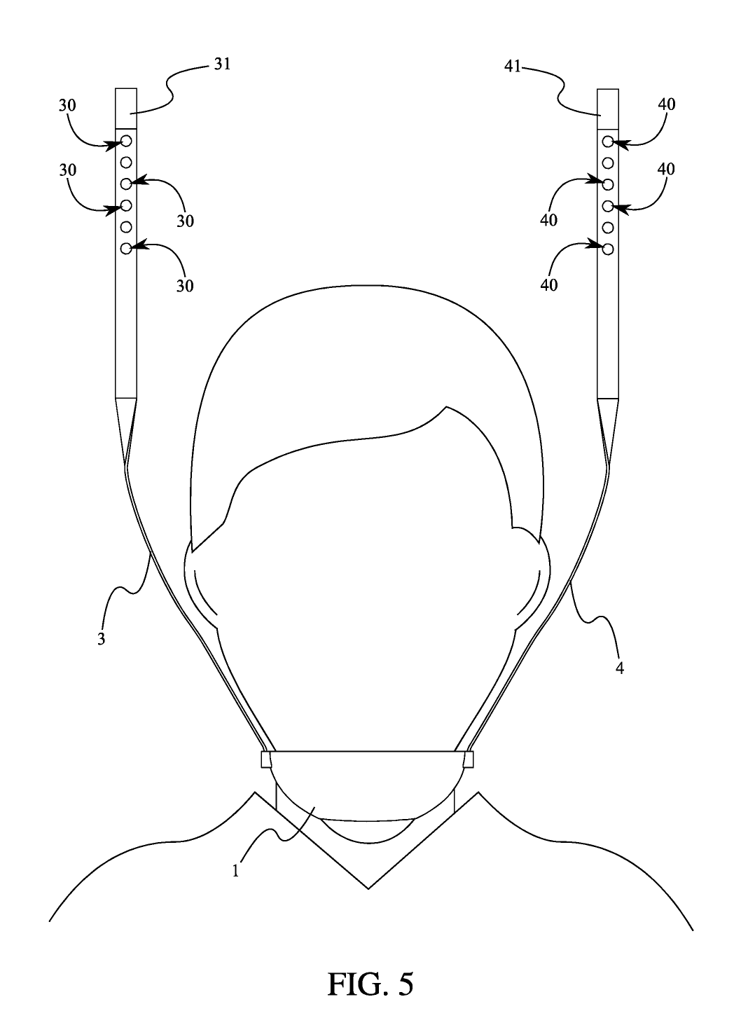

FIG. 6 is a diagram showing the chin cup secured about a patient's chin and the first cranial strap being secured to a medical table; the first fastener being secured to an opposing fastening member of the medical table.

FIG. 7 is a diagram showing the chin cup secured about a patient's chin and the second cranial strap being secured to the medical table; the second fastener being secured to a subsequent fastening member of the medical table.

FIG. 8 is a diagram showing the first cranial strap and the second cranial strap being secured to the medical table, wherein a hook traverses through one of the first plurality of restraining holes and a subsequent hook traverses through one of the second plurality of restraining holes.

FIG. 9 is a front elevational view of the present invention including the third cranial strap.

FIG. 10 is a rear elevational view, showing the adhesive backing of the third cranial strap.

FIG. 11 is a diagram showing the chin cup secured about a patient's chin, and the third cranial strap secured about the patient's forehead.

FIG. 12 is a diagram showing the chin cup secured about a patient's chin, and the first cranial strap and the third cranial strap being secured to a medical table; the first fastener being secured to an opposing fastening member of the medical table and a first hook traversing through one of the fourth plurality of restraining holes.

FIG. 13 is a diagram showing the chin cup secured about a patient's chin, and the second cranial strap and the third cranial strap being secured to the medical table; the second fastener being secured to a subsequent fastening member of the medical table and a second hook traversing through one of the third plurality of restraining holes.

FIG. 14 is a diagram showing the first cranial strap, the second cranial strap, and the third cranial strap being secured to the medical table, wherein a hook traverses through one of the first plurality of restraining holes and a subsequent hook traverses through one of the second plurality of restraining holes, and wherein the third fastener and the fourth fastener are secured to the medical table.

### DETAIL DESCRIPTIONS OF THE INVENTION

All illustrations of the drawings are for the purpose of describing selected versions of the present invention and are not intended to limit the scope of the present invention.

The present invention is a surgical steady chin strap for stabilizing a patient's head during surgery. The present invention is a re-usable or disposable device that is secured about a patient's chin, and optionally secured about the forehead, to apply a force to the patient's head, downwards in the direction of the medical table. The present invention is able to conform to different chin and head sizes, thus allowing the present invention to be used among multiple patients.

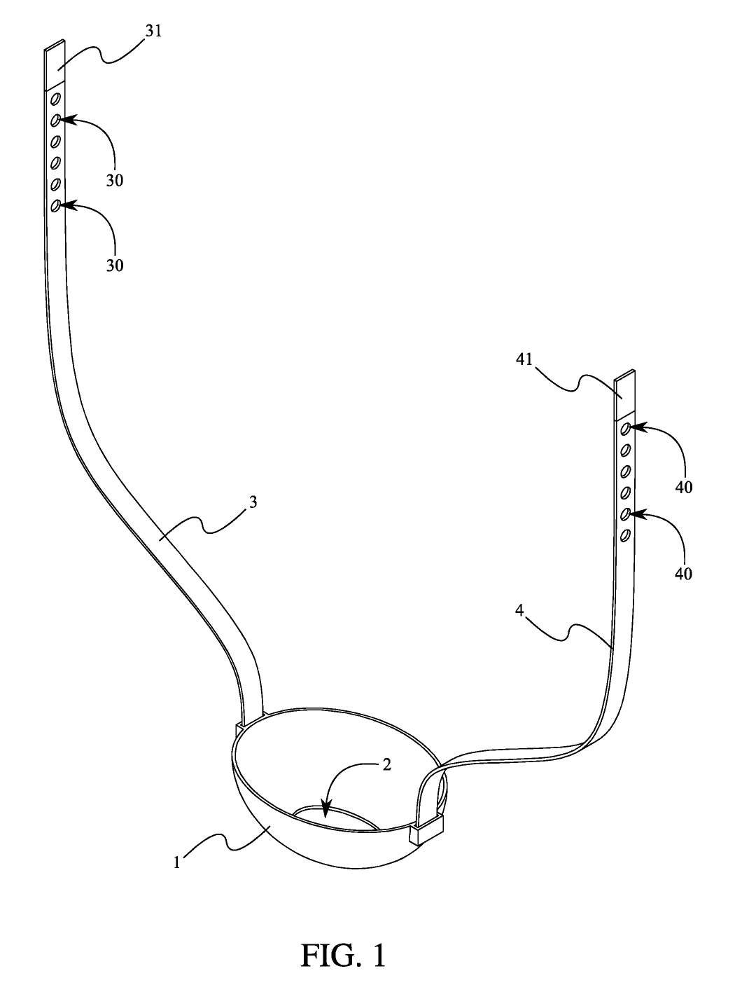

In reference to FIG. 1, the present invention comprises a chin cup 1, a first cranial strap 3, and a second cranial strap 4. The chin cup 1 provides the portion of the present invention that engages with the patient's chin, wherein the chin cup 1 is a concave body that sits around the chin of the user. Meanwhile, the first cranial strap 3 and the second cranial strap 4 provide a pair of anchors to secure the chin cup 1 to the medical table. As such, the chin cup 1 is terminally connected both the first cranial strap 3 and the second cranial strap 4.

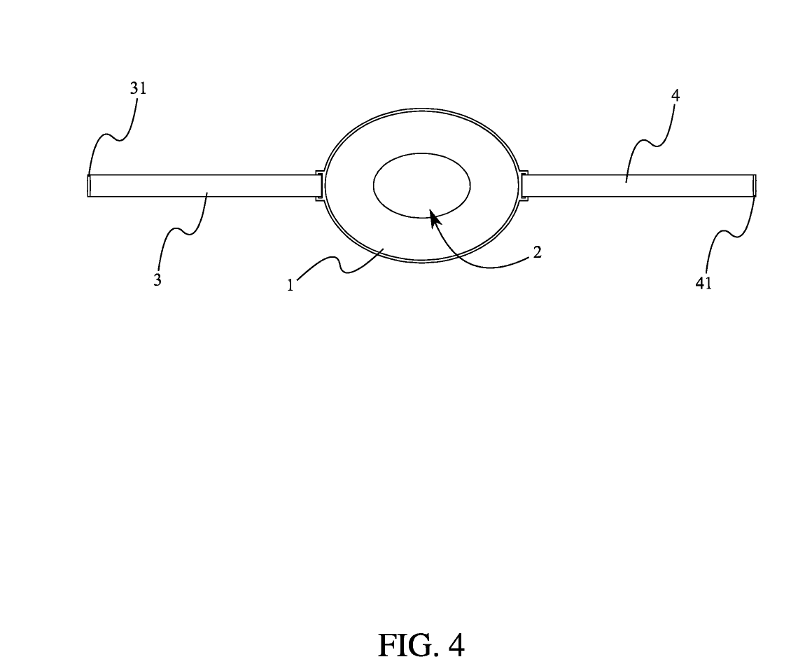

In reference to FIG. 1 and FIG. 4, the first cranial strap 3 and the second cranial strap 4 are positioned opposite each other along the chin cup 1. In this way, the tensile forces enacted by the first cranial strap 3 and the second cranial strap 4 are evenly distributed about the chin cup 1. The first cranial strap 3 runs along one side of the patient's head, while the second cranial strap 4 runs along the other side of the patient's head. Both the first cranial strap 3 and the second cranial strap 4 are secured to the medical table, wherein the first cranial strap 3 and the second cranial strap 4 provide a tensile force to stabilize the chin cup 1 against the patient's chin.

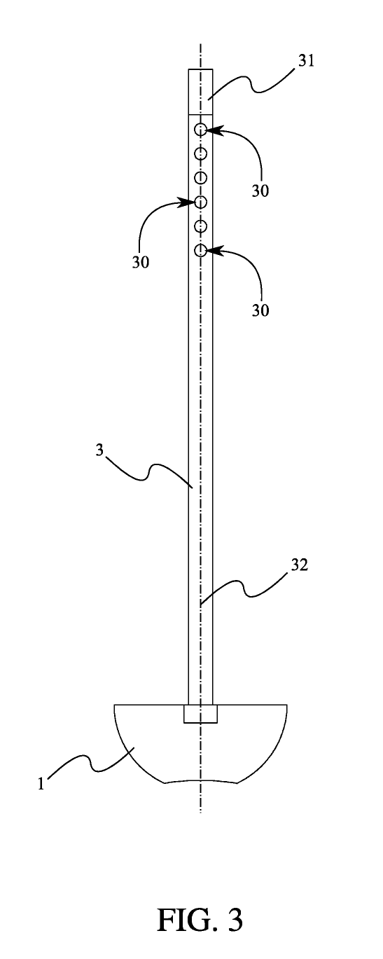

In reference to FIG. 1 and FIG. 3, in order to be secured to the medical table, the first cranial strap 3 comprises a first plurality of restraining holes 30 and a first fastener 31. The first fastener 31 is terminally positioned opposite the chin cup 1, while the first plurality of restraining holes 30 is positioned adjacent to the first fastener 31. Together, the first plurality of restraining holes 30 and the first fastener 31 provide a two factor means for securing the first cranial strap 3 to the medical table. The first plurality of restraining holes 30 allows the first cranial strap 3 to engage with a hook that is positioned along the edge of the medical table. Meanwhile, the first fastener 31 is engaged with an opposing fastening member on the bottom of the medical table in order to secure the terminal end of the first cranial strap 3 beneath the medical table.

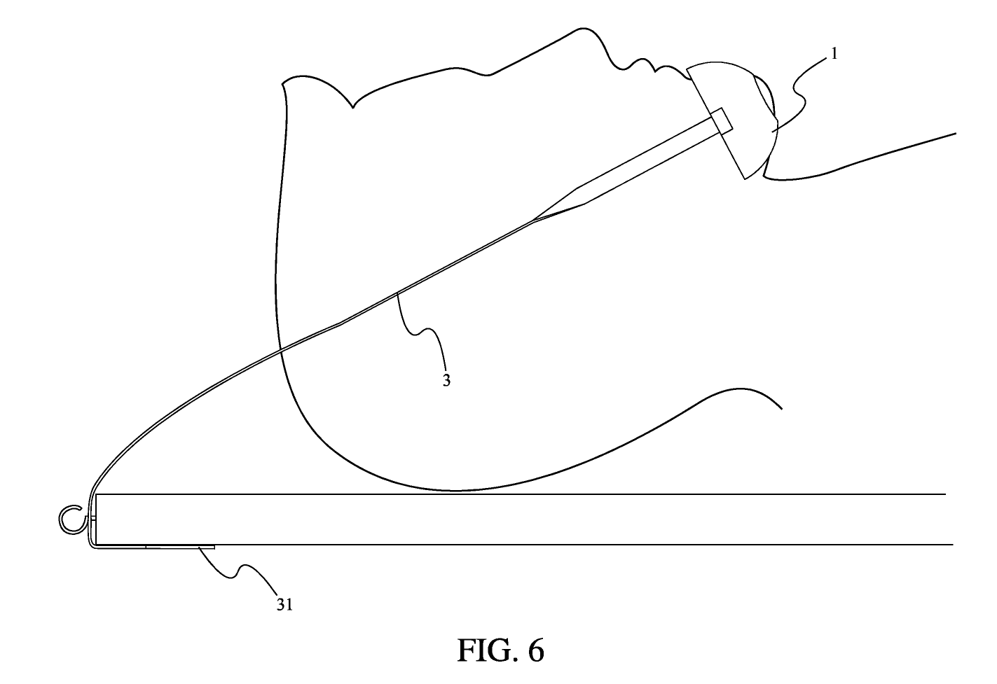

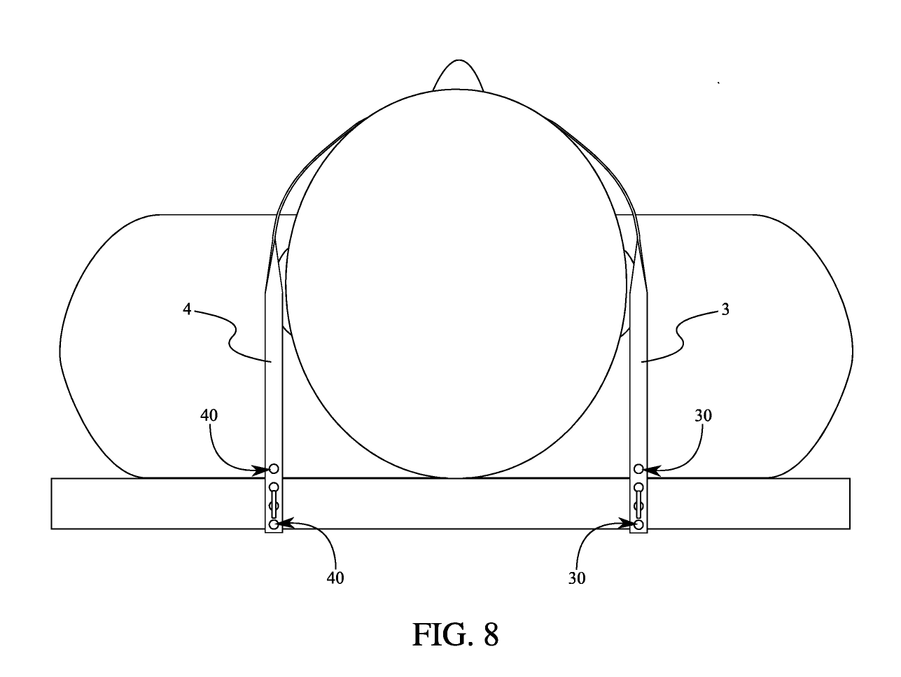

When the chin cup 1 is placed over the patient's chin, the first cranial strap 3 is pulled taught, towards the top of the patient's head and downwards towards the medical table, as depicted in FIG. 6. The first cranial strap 3 is then pulled around the edge of the medical table, such that the hook traverses through one of the first plurality of restraining holes 30, as depicted in FIG. 8. In turn, the first cranial strap 3 is anchored to the medical table at the desired anchor point along the first cranial strap 3. The hole selected from the first plurality of restraining holes 30 for the hook to pass through depends on the desired tautness of the first cranial strap 3. The excess of the first cranial strap 3 is then pulled under the medical table, wherein the first fastener 31 is affixed to the opposing fastening member, as depicted in FIG. 6. The first fastener 31 ensures that the terminal end of the first cranial strap 3 is not free hanging, wherein the first cranial strap 3 could potentially slip off of the hook.

In reference to FIG. 3, each of the first plurality of restraining holes 30 is longitudinally aligned with each other along the body of the first cranial strap 3. The longitudinal alignment of the first plurality of restraining holes 30 allows the first cranial strap 3 to be secured to the medical table at various increments, thus providing different levels of tautness and the ability to accommodate different sized heads. In the preferred embodiment of the present invention, the first plurality of restraining holes 30 is positioned along a longitudinal midline 32 of the first cranial strap 3, as depicted in FIG. 3. This ensures that stress is evenly applied to the body of the first cranial strap 3, when the first cranial strap 3 is secured to the medical table about the hook. Furthermore, in the preferred embodiment of the present invention, the first plurality of restraining holes 30 is equidistantly distributed along the body of the first cranial strap 3. This also ensures that stress is evenly applied to the body of the first cranial strap 3, when the first cranial strap 3 is secured to the medical table about the hook, and allows for more standardized adjustments to be made in varying the tautness of the first cranial strap 3.

In one embodiment of the present invention, the first plurality of restraining holes 30 is linearly arranged along the body of the first cranial strap 3, such that there is a single row of holes positioned along the body of the first cranial strap 3. In other embodiments of the present invention, the first plurality of restraining holes 30 may be longitudinally positioned along the body of the first cranial strap 3 in sets of two or more holes. In such embodiments, the medical table includes one or more additional hooks, such that each hole from the selected set of holes is positioned around a hook. Having multiple attachment points between the first cranial strap 3 and the medical table provides a more secured connection.

In some embodiments, each of the first plurality of restraining holes 30 may be reinforced. The reinforcement of the first plurality of restraining holes 30 gives added strength to the first cranial strap 3, to help prevent the first cranial strap 3 from wearing out prematurely, inadvertently ripping, etc. In one embodiment, each of the first plurality of restraining holes 30 is reinforced using a grommet that is secured to the body of the first cranial strap 3. In another embodiment, a ripstop fabric may be used in the location of each the first plurality of restraining holes 30 in order to provide reinforcement. It is also possible for other methods of reinforcement to be applied in yet other embodiments of the present invention.

In some embodiments of the present invention, the first fastener 31 is selected from the group consisting of a hook fastener and a loop fastener. When the first fastener 31 is selected to be the hook fastener, the opposing fastening member is then selected to be the loop fastener. Conversely, when the first fastener 31 is selected to be the loop fastener, the opposing fastening member is then selected to be the hook fastener. In this way, the first fastener 31 is able to engage with the opposing fastening member to affix the terminal end of the first cranial strap 3 to the medical table, as depicted in FIG. 6. In other embodiments of the present invention, alternative fastening means may be used between the first fastener 31 and the opposing fastening member to secure the first cranial strap 3 to the medical table.

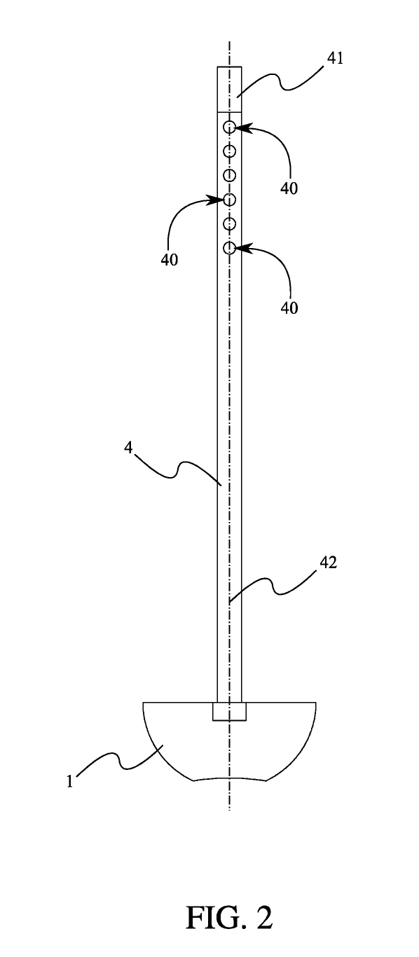

In reference to FIG. 1-2, in order to be secured to the medical table, the second cranial strap 4 comprises a second plurality of restraining holes 40 and a second fastener 41. The second fastener 41 is terminally positioned opposite the chin cup 1, while the second plurality of restraining holes 40 is positioned adjacent to the second fastener 41. Together, the second plurality of restraining holes 40 and the second fastener 41 provide a two factor means for securing the second cranial strap 4 to the medical table. The second plurality of restraining holes 40 allows the second cranial strap 4 to engage with a subsequent hook that is positioned along the edge of the medical table. Meanwhile, the second fastener 41 is engaged with a subsequent opposing fastening member on the bottom of the medical table in order to secure the terminal end of the second cranial strap 4 beneath the medical table.

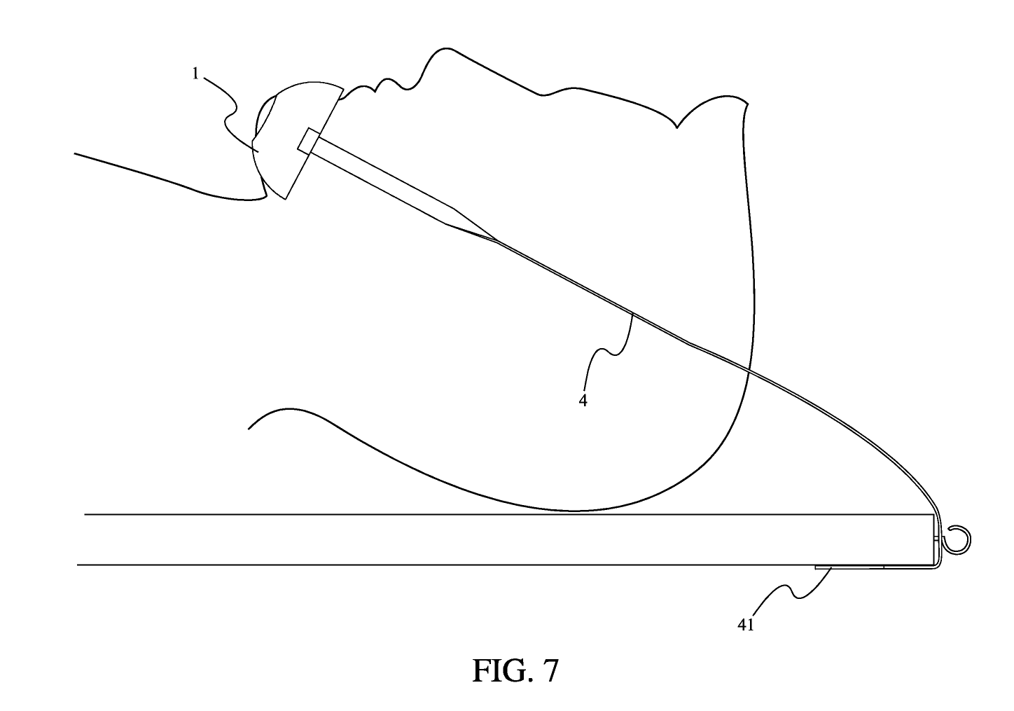

When the chin cup 1 is placed over the patient's chin, the second cranial strap 4 is pulled taught, towards the top of the patient's head and downwards towards the medical table, as depicted in FIG. 7. The second cranial strap 4 is then pulled around the edge of the medical table, such that the subsequent hook traverses through one of the second plurality of restraining holes 40, as depicted in FIG. 8. In turn, the second cranial strap 4 is anchored to the medical table at the desired anchor point along the second cranial strap 4. The hole selected from the second plurality of restraining holes 40 for the subsequent hook to pass through depends on the desired tautness of the second cranial strap 4. The excess of the second cranial strap 4 is then pulled under the medical table, wherein the second fastener 41 is affixed to the subsequent opposing fastening member, as depicted in FIG. 7. The second fastener 41 ensures that the terminal end of the second cranial strap 4 is not free hanging, wherein the second cranial strap 4 could potentially slip off of the subsequent hook.

In reference to FIG. 2, each of the second plurality of restraining holes 40 is longitudinally aligned with each other along the body of the second cranial strap 4. The longitudinal alignment of the second plurality of restraining holes 40 allows the second cranial strap 4 to be secured to the medical table at various increments, thus providing different levels of tautness and the ability to accommodate different sized heads. In the preferred embodiment of the present invention, the second plurality of restraining holes 40 is positioned along a longitudinal midline 42 of the second cranial strap 4, as depicted in FIG. 2. This ensures that stress is evenly applied to the body of the second cranial strap 4, when the second cranial strap 4 is secured to the medical table about the subsequent hook. Furthermore, in the preferred embodiment of the present invention, the second plurality of restraining holes 40 is equidistantly distributed along the body of the second cranial strap 4. This also ensures that stress is evenly applied to the body of the second cranial strap 4, when the second cranial strap 4 is secured to the medical table about the subsequent hook, and allows for more standardized adjustments to be made in varying the tautness of the second cranial strap 4.

In one embodiment of the present invention, the second plurality of restraining holes 40 is linearly arranged along the body of the second cranial strap 4, such that there is a single row of holes positioned along the body of the second cranial strap 4. In other embodiments of the present invention, the second plurality of restraining holes 40 may be longitudinally positioned along the body of the second cranial strap 4 in sets of two or more holes. In such embodiments, the medical table includes one or more additional subsequent hooks, such that each hole from the selected set of holes is positioned around a hook. Having multiple attachment points between the second cranial strap 4 and the medical table provides a more secured connection.

In some embodiments, each of the second plurality of restraining holes 40 may be reinforced. The reinforcement of the second plurality of restraining holes 40 gives added strength to the second cranial strap 4, to help prevent the second cranial strap 4 from wearing out prematurely, inadvertently ripping, etc. In one embodiment, each of the second plurality of restraining holes 40 is reinforced using a grommet that is secured to the body of the second cranial strap 4. In another embodiment, a ripstop fabric may be used in the location of each the second plurality of restraining holes 40 in order to provide reinforcement. It is also possible for other methods of reinforcement to be applied in yet other embodiments of the present invention.

In some embodiments of the present invention, the second fastener 41 is selected from the group consisting of a hook fastener and a loop fastener. When the second fastener 41 is selected to be the hook fastener, the subsequent opposing fastening member is then selected to be the loop fastener. Conversely, when the second fastener 41 is selected to be the loop fastener, the subsequent opposing fastening member is then selected to be the hook fastener. In this way, the second fastener 41 is able to engage with the subsequent opposing fastening member to affix the terminal end of the second cranial strap 4 to the medical table, as depicted in FIG. 7. In other embodiments of the present invention, alternative fastening means may be used between the second fastener 41 and the subsequent opposing fastening member to secure the second cranial strap 4 to the medical table.

In reference to FIG. 1 and FIG. 4, in some embodiments, the present invention may further comprise a chin hole 2. The chin hole 2 traverses through the chin cup 1, thus allowing a portion of the patient's chin to protrude through the chin cup 1. Ideally, the chin hole 2 is centrally positioned through the chin cup 1, as depicted in FIG. 4, in order to best accommodate the passage of the patient's chin through the chin cup 1.

Additionally, the central positioning of the chin hole 2 through the chin cup 1 ensures that stress is evenly distributed about the chin cup 1 when the first cranial strap 3 and the second cranial strap 4 are pulled tight. The chin hole 2 allows for more contact between the patient's chin and the chin cup 1 in many situations, as the sides of the chin cup 1 are then able to better conform to the shape of the patient's face. Furthermore, the chin hole 2 allows the chin cup 1 to be used with a greater number of patients, as the chin cup 1 is more able to conform to different sized chins.

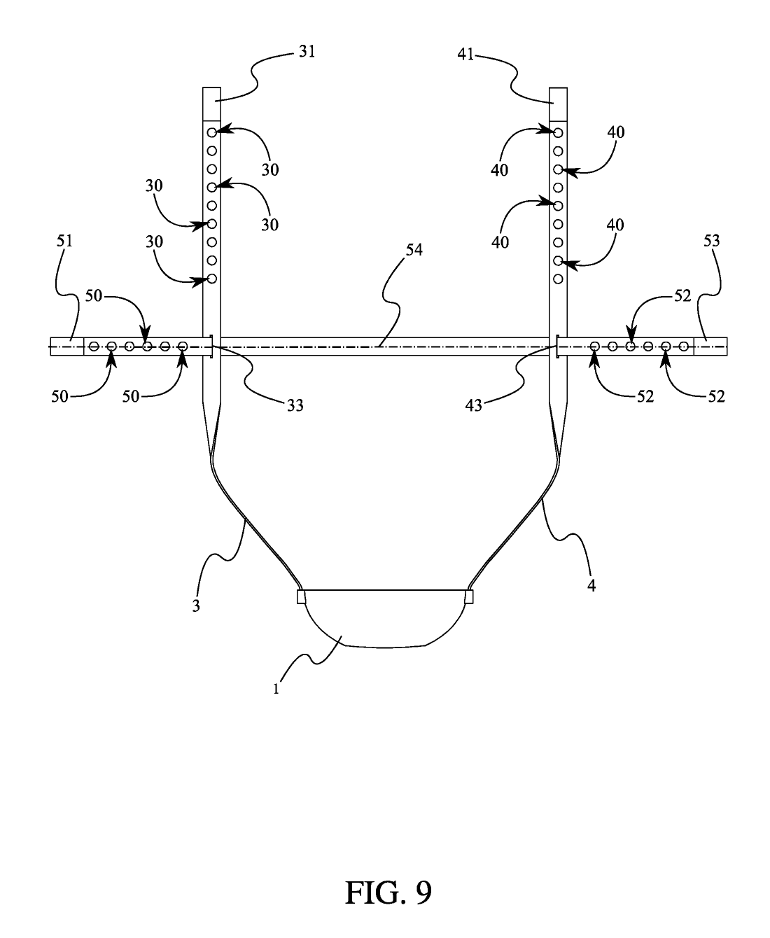

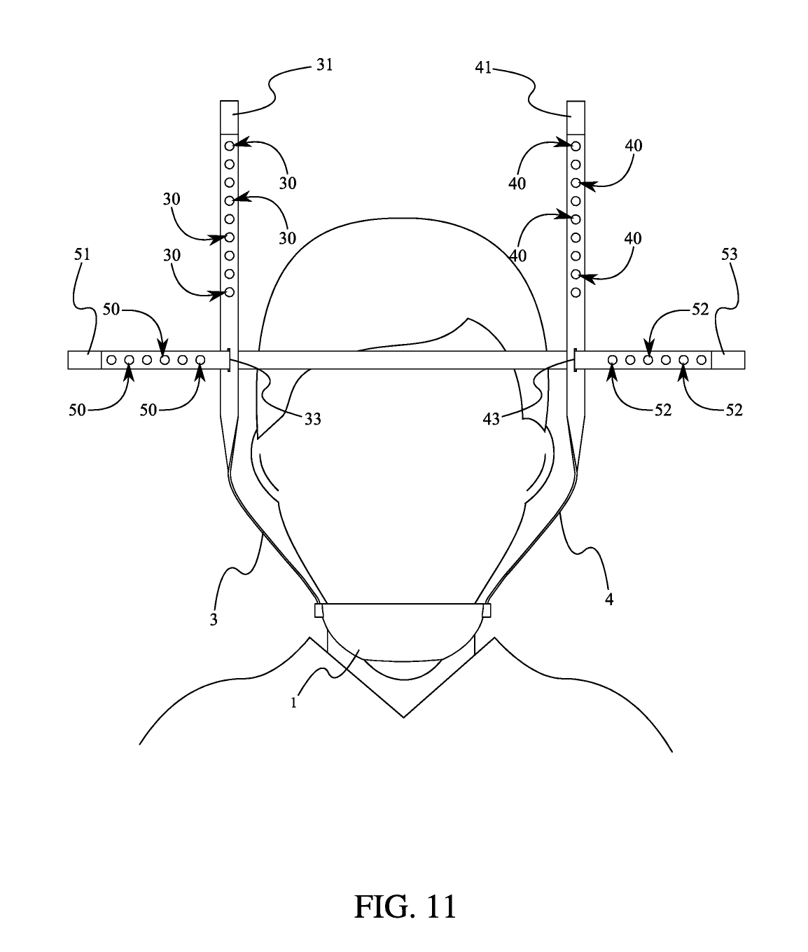

In reference to FIG. 9, in some embodiments, the present invention may further comprise a third cranial strap 5 that further assists in securing the patient's head about the medical table. The third cranial strap 5 traverses through the first cranial strap 3 and the second cranial strap 4, wherein the third cranial strap 5 runs perpendicular to both the first cranial strap 3 and the second cranial strap 4. The third cranial strap 5 is positioned in between the chin cup 1 and both the first plurality of restraining holes 30 and the second plurality of restraining holes 40. Furthermore, the third cranial strap 5 is positioned along the first cranial strap 3 and the second cranial strap 4, such that the third cranial strap 5 rests across the patient's forehead, when the present invention is secured to the patient's head, as depicted in FIG. 11.

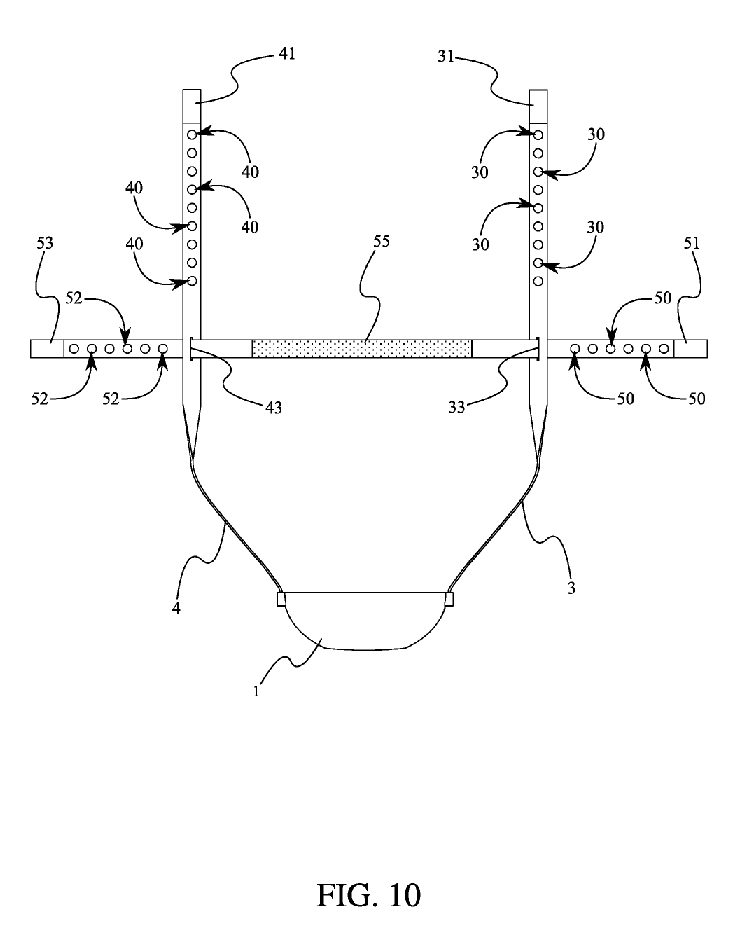

In reference to FIG. 9-10, in some embodiments of the present invention, the first cranial strap 3 comprises a first slit 33, while the second cranial strap 4 comprises a second slit 43. The first slit 33 is made along the length of the body of the first cranial strap 3, while the second slit 43 is made along the length of the body of the second cranial strap 4. Ideally, the first slit 33 is made along the longitudinal midline 32 of the first cranial strap 3 and second slit 43 is made along the longitudinal midline 42 of the second cranial strap 4 to preserve the structural stability of the first cranial strap 3 and the second cranial strap 4, respectively. The first slit 33 allows the third cranial strap 5 to traverse through the first cranial strap 3, while the second slit 43 allows the third cranial strap 5 to traverse through the second cranial strap 4.

In other embodiments of the present invention, the first cranial strap 3 may comprise a first plurality of slits, wherein the first plurality of slits is oriented along the length of the body of the first cranial strap 3. may be made along the length of the first cranial strap 3 and the second cranial strap 4. Simultaneously, the second cranial strap 4 may comprise a second plurality of slits, wherein the second plurality of slits is oriented along the length of the body of the second cranial strap 4. Together, the first plurality of slits and the second plurality of slits allow the position of the third cranial strap 5 to be varied along the first cranial strap 3 and the second cranial strap 4, in order to accommodate different head sizes.

In some embodiments, the first slit 33 or the first plurality of slits may be reinforced. The reinforcement of the first slit 33 or the first plurality of slits gives added strength to the first cranial strap 3, to help prevent the first cranial strap 3 from wearing out prematurely, inadvertently ripping, etc. In one embodiment, the first slit 33 or each of the first plurality of slits is reinforced using a grommet that is secured to the body of the first cranial strap 3. In another embodiment, a ripstop fabric may be used in the location of the first slit 33 or the first plurality of slits in order to provide reinforcement. It is also possible for other methods of reinforcement to be applied in yet other embodiments of the present invention.

In some embodiments, the second slit 43 or the second plurality of slits may be reinforced. The reinforcement of the second slit 43 or the second plurality of slits gives added strength to the second cranial strap 4, to help prevent the second cranial strap 4 from wearing out prematurely, inadvertently ripping, etc. In one embodiment, the second slit 43 or each of the second plurality of slits is reinforced using a grommet that is secured to the body of the second cranial strap 4. In another embodiment, a ripstop fabric may be used in the location of the second slit 43 or the second plurality of slits in order to provide reinforcement. It is also possible for other methods of reinforcement to be applied in yet other embodiments of the present invention.

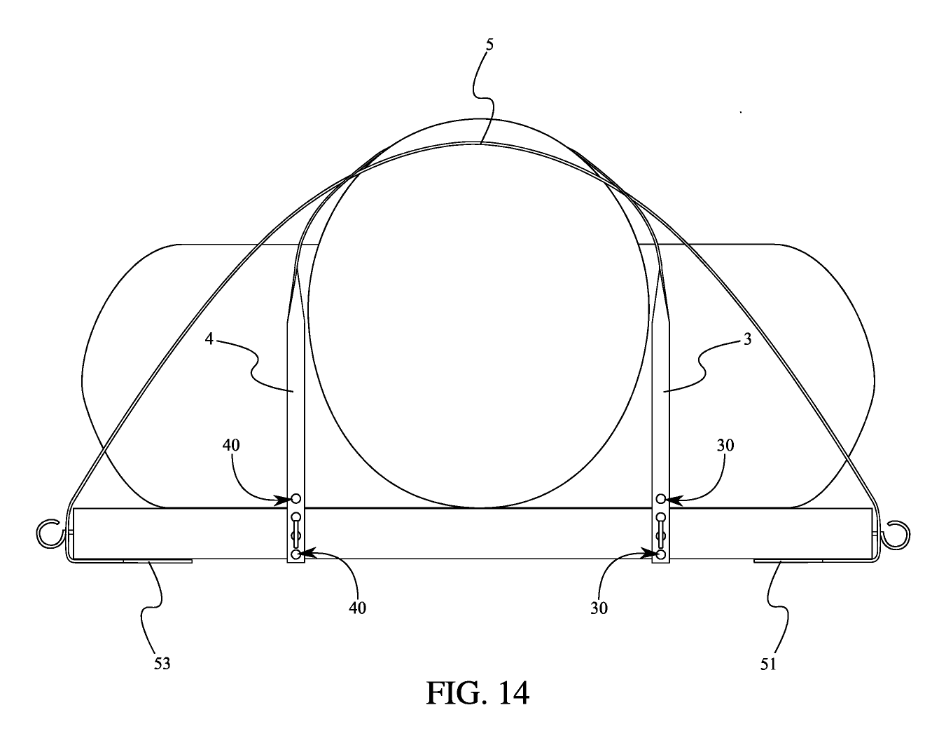

In reference to FIG. 9-10, in order to secure the third cranial strap 5 to the medical table, the third cranial strap 5 comprises a third fastener 51 and a fourth fastener 53, wherein the third fastener 51 and the fourth fastener 53 are terminally positioned opposite each other along the body of the third cranial strap 5. The third fastener 51 and the fourth fastener 53 are engaged with a pair of opposing fastening members on the bottom of the medical table in order to secure the terminal ends of the third cranial strap 5 beneath the medical table. As such, each of the pair of opposing fastening members is positioned about an opposing side of the medical table. When the third cranial strap 5 is placed over the patient's forehead, the third cranial strap 5 is pulled taught, downwards towards the medical table. Both ends of the third cranial strap 5 are then pulled around the side edges of the medical table, wherein the third fastener 51 and the fourth fastener 53 are affixed to the pair of opposing fastening members, as depicted in FIG. 14.

In some embodiments of the present invention, the third fastener 51 is selected from the group consisting of a hook fastener and a loop fastener. When the third fastener 51 is selected to be the hook fastener, a first fastening member from the pair of opposing fastening members is then selected to be the loop fastener. Conversely, when the third fastener 51 is selected to be the loop fastener, the first fastening member is then selected to be the hook fastener. In this way, the third fastener 51 is able to engage with the subsequent opposing fastening member to affix the terminal end of the third cranial strap 5 to the medical table, as depicted in FIG. 14. In other embodiments of the present invention, alternative fastening means may be used between the third fastener 51 and the first fastening member to secure the third cranial strap 5 to the medical table.

Similarly, in some embodiments of the present invention, the fourth fastener 53 is selected from the group consisting of a hook fastener and a loop fastener. When the fourth fastener 53 is selected to be the hook fastener, a second fastening member from the pair of opposing fastening members is then selected to be the loop fastener. Conversely, when the fourth fastener 53 is selected to be the loop fastener, the second fastening member is then selected to be the hook fastener. In this way, the fourth fastener 53 is able to engage with the second fastening member to affix the terminal end of the third cranial strap 5 to the medical table, as depicted in FIG. 14. In other embodiments of the present invention, alternative fastening means may be used between the fourth fastener 53 and the second fastening member to secure the third cranial strap 5 to the medical table.

In reference to FIG. 9-11, in some embodiments of the present invention, the third cranial strap 5 may further comprise a third plurality of restraining holes 50 and a fourth plurality of restraining holes 52 to further secure the third cranial strap 5 to the medical table. The third plurality of restraining holes 50 is positioned adjacent to the third fastener 51, while the fourth plurality of restraining holes 52 is positioned adjacent to the fourth fastener 53. Together, the third plurality of restraining holes 50 and the third fastener 51 provide a two factor means for securing one end of the third cranial strap 5 to the medical table. Similarly, the fourth plurality of restraining holes 52 and the fourth fastener 53 work together to provide a two factor means for securing the other end of the third cranial strap 5 to the medical table.

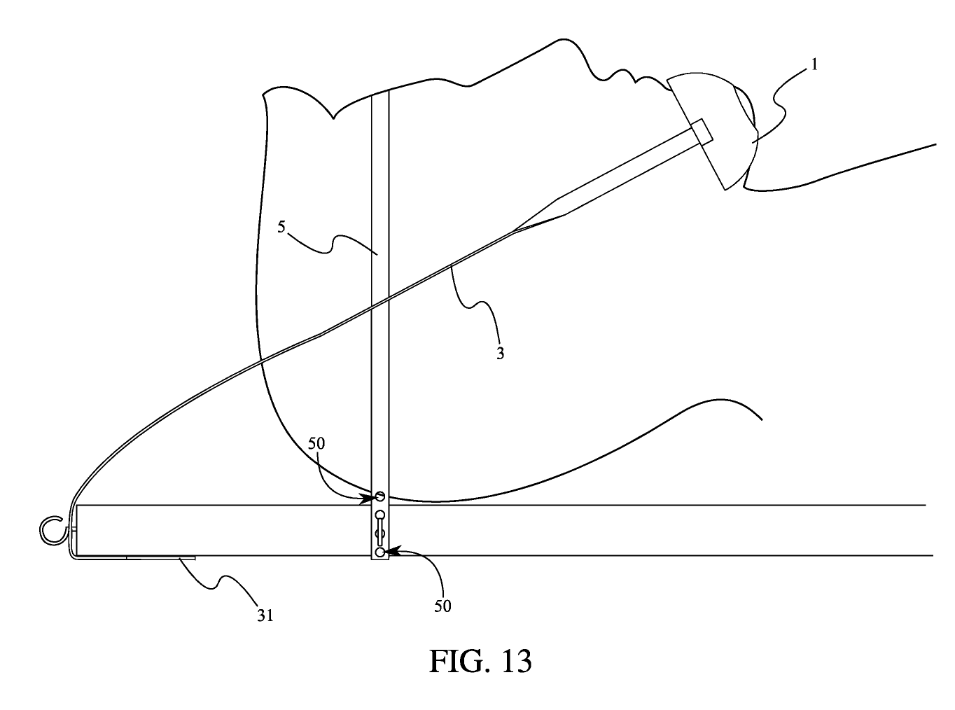

The third plurality of restraining holes 50 and the fourth plurality of restraining holes 52 allow the third cranial strap 5 to engage with a pair of opposing hooks positioned along the side edges of the medical table; each of the pair of opposing hooks being positioned on an opposing side of the medical table. The third plurality of restraining holes 50 allows the third cranial strap 5 to engage with a first hook from the pair of opposing hooks that is positioned along one side edge of the medical table, as depicted in FIG. 13-14. Similarly, the fourth plurality of restraining holes 52 allows the third cranial strap 5 to engage with a second hook from the pair of opposing hooks that is positioned along the opposite side edge of the medical table, as depicted in FIG. 13 and FIG. 14. Meanwhile, the third fastener 51 and the fourth fastener 53 are engaged with the pair of opposing fastening members on the bottom of the medical table in order to further secure the terminal ends of the third cranial strap 5 beneath the medical table.

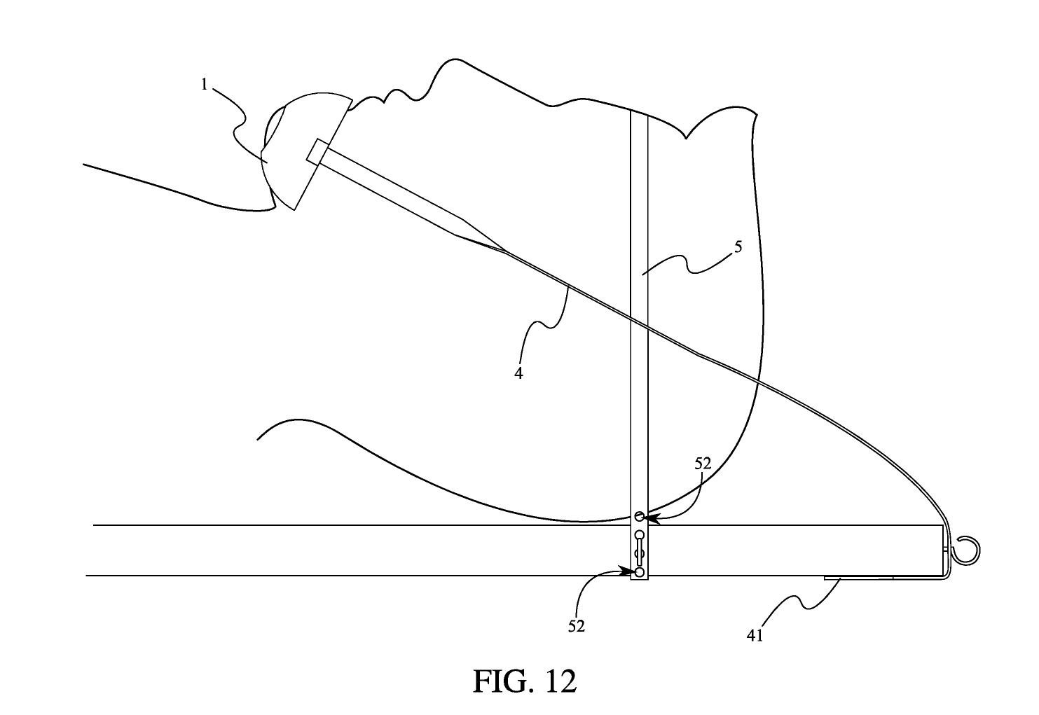

In reference to FIG. 12-14, when the third cranial strap 5 is placed over the patient's forehead, the third cranial strap 5 is pulled taught, downwards towards the medical table. The third cranial strap 5 is then pulled around the side edges of the medical table, such that the first hook traverses through one of the third plurality of restraining holes 50 and the second hook traverses through one of the fourth plurality of restraining holes 52. In turn, the third cranial strap 5 is anchored to the medical table at the desired anchor points along the third cranial strap 5. The hole selected from the third plurality of restraining holes 50 for the first hook to pass through, and the hole selected from the fourth plurality of restraining holes 52 for the second hook to pass through, depend on the desired tautness of the third cranial strap 5. The excess of the third cranial strap 5 is then pulled under the medical table, wherein the third fastener 51 is affixed to the first fastening member and the fourth fastener 53 is affixed to the second fastening member. The third fastener 51 and the fourth fastener 53 ensure that the terminal ends of the third cranial strap 5 are not free hanging, wherein the third cranial strap 5 could potentially slip off of either of the pair of opposing hooks.

In reference to FIG. 9-11, each of the third plurality of restraining holes 50 is longitudinally aligned with each other along the body of the third cranial strap 5. The longitudinal alignment of the third plurality of restraining holes 50 allows the third cranial strap 5 to be secured to the medical table at various increments, thus providing different levels of tautness and the ability to accommodate different sized heads. In the preferred embodiment of the present invention, the third plurality of restraining holes 50 is positioned along a longitudinal midline 54 of the third cranial strap 5, as depicted in FIG. 9. This ensures that stress is evenly applied to the body of the third cranial strap 5, when the third cranial strap 5 is secured to the medical table about the first hook. Furthermore, in the preferred embodiment of the present invention, the third plurality of restraining holes 50 is equidistantly distributed along the body of the third cranial strap 5. This also ensures that stress is evenly applied to the body of the third cranial strap 5, when the third cranial strap 5 is secured to the medical table about the subsequent hook, and allows for more standardized adjustments to be made in varying the tautness of the third cranial strap 5.

In one embodiment of the present invention, the third plurality of restraining holes 50 is linearly arranged along the body of the third cranial strap 5, such that there is a single row of holes positioned along one end of the body of the third cranial strap 5. In other embodiments of the present invention, the third plurality of restraining holes 50 may be longitudinally positioned along the body of the third cranial strap 5 in sets of two or more holes. In such embodiments, the medical table includes one or more additional first hooks, such that each hole from the selected set of holes is positioned around a hook. Having multiple attachment points between the third cranial strap 5 and the medical table provides a more secured connection.

In some embodiments, each of the third plurality of restraining holes 50 may be reinforced. The reinforcement of the third plurality of restraining holes 50 gives added strength to the third cranial strap 5, to help prevent the third cranial strap 5 from wearing out prematurely, inadvertently ripping, etc. In one embodiment, each of the third plurality of restraining holes 50 is reinforced using a grommet that is secured to the body of the third cranial strap 5. In another embodiment, a ripstop fabric may be used in the location of each the third plurality of restraining holes 50 in order to provide reinforcement. It is also possible for other methods of reinforcement to be applied in yet other embodiments of the present invention.

Similar to the third plurality of restraining holes 50 and in reference to FIG. 9-11, each of the fourth plurality of restraining holes 52 is longitudinally aligned with each other along the body of the third cranial strap 5. The longitudinal alignment of the fourth plurality of restraining holes 52 allows the third cranial strap 5 to be secured to the medical table at various increments, thus providing different levels of tautness and the ability to accommodate different sized heads. In the preferred embodiment of the present invention, the fourth plurality of restraining holes 52 is positioned along the longitudinal midline 54 of the third cranial strap 5, as depicted in FIG. 9. This ensures that stress is evenly applied to the body of the third cranial strap 5, when the third cranial strap 5 is secured to the medical table about the second hook. Furthermore, in the preferred embodiment of the present invention, the fourth plurality of restraining holes 52 is equidistantly distributed along the body of the third cranial strap 5. This also ensures that stress is evenly applied to the body of the third cranial strap 5, when the third cranial strap 5 is secured to the medical table about the second hook, and allows for more standardized adjustments to be made in varying the tautness of the third cranial strap 5.

In one embodiment of the present invention, the fourth plurality of restraining holes 52 is linearly arranged along the body of the third cranial strap 5, such that there is a single row of holes positioned along the other end of the body of the third cranial strap 5. In other embodiments of the present invention, the fourth plurality of restraining holes 52 may be longitudinally positioned along the body of the third cranial strap 5 in sets of two or more holes. In such embodiments, the medical table includes one or more additional second hooks, such that each hole from the selected set of holes is positioned around a hook. Having multiple attachment points between the third cranial strap 5 and the medical table provides a more secured connection.

In some embodiments, each of the fourth plurality of restraining holes 52 may be reinforced. The reinforcement of the fourth plurality of restraining holes 52 gives added strength to the third cranial strap 5, to help prevent the third cranial strap 5 from wearing out prematurely, inadvertently ripping, etc. In one embodiment, each of the fourth plurality of restraining holes 52 is reinforced using a grommet that is secured to the body of the third cranial strap 5. In another embodiment, a ripstop fabric may be used in the location of each the fourth plurality of restraining holes 52 in order to provide reinforcement. It is also possible for other methods of reinforcement to be applied in yet other embodiments of the present invention.

In reference to FIG. 10, in some embodiments of the present invention, the third cranial strap 5 may further comprise an adhesive backing 55. The adhesive backing 55 is positioned in between the first cranial strap 3 and the second cranial strap 4 and oriented to face the forehead of the patient. When the present invention is secured to the user, the chin cup 1 is first positioned around the patient's chin. As the first cranial strap 3, the second cranial strap 4, and the third strap are pulled downwards to be secured to the medical table, the adhesive backing 55 is pressed against the patient's forehead. The first cranial strap 3, the second cranial strap 4, and the third strap are then secured to the medical table. Meanwhile, the adhesive backing 55 prevents the patient's head from sliding along the third cranial strap 5, thus retaining the patient's head in a stable position.

The present invention can be configured for single use or configured to be re-usable. In embodiments where the present invention is configured for single use, the first cranial strap 3, the second cranial strap 4, and the third cranial strap 5 may be constructed from a biodegradable plastic, or other biodegradable material, or a recyclable material. Furthermore, the chin cup 1 may be constructed from a recycled cup holder material, or other recyclable material, or a biodegradable material. The user of recyclable or biodegradable materials in single use variations of the present invention acts to reduce the environmental impact of the disposal of the present invention.

Although the invention has been explained in relation to its preferred embodiment, it is to be understood that many other possible modifications and variations can be made without departing from the spirit and scope of the invention as hereinafter claimed.

What is claimed is:

1. A surgical steady chin strap comprises:
a chin cup being terminally connected to both a first cranial strap and a second cranial strap;
the first cranial strap and the second cranial strap being positioned opposite each other along the chin cup;
the first cranial strap comprising a first plurality of restraining holes and a first fastener;
the first fastener being terminally positioned opposite the chin cup;
the first plurality of restraining holes being positioned adjacent to the first fastener;
the first plurality of restraining holes being longitudinally aligned with each other;
the second cranial strap comprising a second plurality of restraining holes and a second fastener;
the second fastener being terminally positioned opposite the chin cup;
the second plurality of restraining holes being positioned adjacent to the second fastener; and
the second plurality of restraining holes being longitudinally aligned with each other.

2. The surgical steady chin strap as claimed in claim 1 comprises:
a chin hole; and
the chin hole traversing through the chin cup.

3. The surgical steady chin strap as claimed in claim 2 comprises:
the chin hole being centrally positioned through the chin cup.

4. The surgical steady chin strap as claimed in claim 1 comprises:
the first fastener being selected from the group consisting of a hook fastener and a loop fastener.

5. The surgical steady chin strap as claimed in claim 1 comprises:
the first plurality of restraining holes being positioned along a longitudinal midline of the first cranial strap.

6. The surgical steady chin strap as claimed in claim 1 comprises:
the first plurality of restraining holes being equidistantly distributed.

7. The surgical steady chin strap as claimed in claim 1 comprises:
the second fastener being selected from the group consisting of a hook fastener and a loop fastener.

8. The surgical steady chin strap as claimed in claim 1 comprises:
the second plurality of restraining holes being positioned along a longitudinal midline of the second cranial strap.

9. The surgical steady chin strap as claimed in claim 1 comprises:
the second plurality of restraining holes being equidistantly distributed.

10. The surgical steady chin strap as claimed in claim 1 comprises:
a third cranial strap;
the third cranial strap traversing through the first cranial strap and the second cranial strap; and
the third cranial strap being positioned in between the chin cup and both the first plurality of restraining holes and the second plurality of restraining holes.

11. The surgical steady chin strap as claimed in claim 10 comprises:
the third cranial strap comprising a third fastener and a fourth fastener; and
the third fastener and the fourth fastener being terminally positioned opposite each other.

12. The surgical steady chin strap as claimed in claim 11 comprises:
the third fastener being selected from the group consisting of a hook fastener and a loop fastener.

13. The surgical steady chin strap as claimed in claim 11 comprises:
the fourth fastener being selected from the group consisting of a hook fastener and a loop fastener.

14. The surgical steady chin strap as claimed in claim 11 comprises:
the third cranial strap further comprising a third plurality of restraining holes and a fourth plurality of restraining holes;
the third plurality of restraining holes being positioned adjacent to the third fastener; and
the fourth plurality of restraining holes being positioned adjacent to the fourth fastener.

15. The surgical steady chin strap as claimed in claim 14 comprises:
the third plurality of restraining holes being positioned along a longitudinal midline of the third cranial strap; and
the fourth plurality of restraining holes being positioned along the longitudinal midline.

16. The surgical steady chin strap as claimed in claim 14 comprises:
the third plurality of restraining holes being longitudinally aligned with each other; and
the third plurality of restraining holes being equidistantly distributed.

17. The surgical steady chin strap as claimed in claim 14 comprises:
the fourth plurality of restraining holes being longitudinally aligned with each other; and
the fourth plurality of restraining holes being equidistantly distributed.

18. The surgical steady chin strap as claimed in claim 10 comprises:
the first cranial strap further comprising a first slit;
the first slit being positioned adjacent the to the first plurality of restraining holes, opposite the first fastener; and
the third cranial strap traversing through the first slit.

19. The surgical steady chin strap as claimed in claim 10 comprises:
the second cranial strap further comprising a second slit;
the second slit being positioned adjacent the to the second plurality of restraining holes, opposite the second fastener; and
the third cranial strap traversing through the second slit.

20. The surgical steady chin strap as claimed in claim 10 comprises:
the third cranial strap comprising an adhesive backing; and
the adhesive backing being positioned in between the first cranial strap and the second cranial strap.
